(19) United States
(12) Patent Application Publication
(10) Pub. No.: US 2020/0188287 A1
(43) Pub. Date: Jun. 18, 2020

(54) COSMETIC AGENTS FOR CONDITIONING KERATIN FIBRES

(71) Applicant: Henkel AG & Co. KGaA, Duesseldorf (DE)

(72) Inventors: TINA AHNFELDT, Hamburg (DE); SYLVIA KERL, Boenningstedt (DE)

(73) Assignee: Henkel AG & Co. KGaA, Duesseldorf (DE)

(21) Appl. No.: 16/694,430

(22) Filed: Nov. 25, 2019

(30) Foreign Application Priority Data
Dec. 17, 2018 (DE) 10 2018 221 938.9

(51) Int. Cl. A61K 8/92; A61K 8/37; A61K 8/31; A61K 8/42; A61Q 5/12

(52) U.S. Cl. CPC A61K 8/922; A61K 8/375; A61Q 5/12; A61K 8/42; A61K 8/31

(57) ABSTRACT

The present disclosure relates to a silicone-free cosmetic agent comprising: one or more polyamides, one or more esters; Ricinus Communis (Castor) Seed Oil, isostearyl alcohol, or a mixture thereof; and one or more hydrocarbons, and use thereof for the care of keratin fibres.

### CROSS-REFERENCE TO RELATED APPLICATION

This application claims priority to German Patent Application No. 10 2018 221 938.9, filed Dec. 17, 2018, which is incorporated herein by reference in its entirety.

### TECHNICAL FIELD

The present disclosure relates to cosmetic agents which comprise either Ricinus Communis (Castor) Seed Oil or isostearyl alcohol or a mixture thereof; together with polyamides, esters and hydrocarbons, and are free from silicones, and use thereof for the care of keratin fibres. The cosmetic agents are particularly preferred in the form of an oil.

### BACKGROUND

Hair conditioners having a caring effect consist primarily of quats (quaternary ammonium compounds), silicone oils, fatty acids and polymer(s). For a few years now, the field of hair cosmetics has been developing hair oils that consist primarily of silicone oils. In addition, the hair oils are used as “leave-in products”, that is to say the formulations remain in the hair without subsequent rinsing. The consumer thus saves time and water. The hair oils enjoy an advantageous sustainability from this point of view. The success of hair oils of this kind on the market has been very high.

The search is ongoing for new combinations that can ensure a high level of care, a perceptible care effect, and good stability. Within the scope of sustainability, increasing value is currently being placed on raw materials of plant origin or having good biodegradability. In this regard, silicone oils are a constant source of criticism. The good level of care, however, should not be compromised. The greatest difficulty in replacing silicone oils in hair oils lies in finding natural oils or natural raw materials that do not weigh down the hair or make it greasy.

Besides natural ambient influences, human hair is also exposed to a range of further, in particular cosmetic stresses. These stresses, which burden the hair, for example include hair dyeing and hair shaping, for example as a result of permanent waving. Cosmetic haircare agents are used in order to reduce the disadvantageous effects of the (ambient) influences that compromise the hair structure, but also in order to maintain and improve the natural hair structure. An essential active substance in many of these cosmetic agents is constituted by the organosilicon compounds, in particular the silicones such as trisiloxanes, which are exemplified by caring properties. The disadvantages of these silicones are the reduced penetration of active substances and auxiliaries into the hair caused by wetting of the hair surface, and the increased difficulty in styling the hair, likewise caused by the wetting of the hair surface. The provision of care agents that have a low silicone content or are free from silicone is therefore a relevant objective in the field of hair cosmetics.

The user of ester oils in hair-conditioning agents is described in international patent application WO 2010/063565 A1 (Henkel).

Document EP 2 623 088 A2 (Henkel) discloses haircare agents that contain at least one ester oil and at least one isoparaffin from the group of isodecane, isoundecane, isododecane, isotridecane and isotetradecane. Mixtures of ester oils and polymer quaternary ammonium compounds are additionally described, as are also combinations of both cosmetic agents.

### BRIEF SUMMARY

This disclosure provides a silicone-free cosmetic agent comprising:

a) one or more polyamides,
b) one or more esters,
c) Ricinus Communis (Castor) Seed Oil, isostearyl alcohol, or a mixture thereof, and
d) one or more hydrocarbons.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure is to provide cosmetic agents that make the co-use of organosilicon compounds, in particular silicones, superfluous and yet nevertheless maintain the performance range of the silicone-containing formulations. The weighing-down of the hair and the greasy condition of the treated hair often occurring with the use of silicones should be avoided. In particular, the object of the present disclosure is to enable the use of renewable raw materials in a silicone-free cosmetic agent, while maintaining beneficial care and conditioning properties when the agent is used to treat hair fibres.

The object is achieved as contemplated herein by a silicone-free cosmetic agent comprising:

a) one or more polyamides,
b) one or more esters,
c) Ricinus Communis (Castor) Seed Oil or isostearyl alcohol or a mixture thereof, and
d) one or more hydrocarbons.

It has been found as contemplated herein that the combination of the above components gives rise to a cosmetic agent, in particular a hair oil, that, in use, is advantageous in terms of silky hair feel, enhancement of shine/gloss, and beneficial smoothness, softness, and detangling.

In particular, cosmetic agents that surprisingly provide improved care in respect of their detangling ability and combability, feel, shine and anti-frizz properties, without weighing down the hair, are produced. Especially by the use of Ricinus Communis (Castor) Seed Oil, the cosmetic agent as contemplated herein gains even more advantages in respect of its care properties. In addition, there is a strong sustainable aspect to the present disclosure as it enables the incorporation of plant oils based on renewable raw materials. In particular, the use of plant-based alkanes is possible, whereby the use of petrochemical constituents can be reduced or avoided.

As component (a), the cosmetic agents include at least one polyamide. It will be understood by the skilled person that the polyamide must be cosmetically acceptable, i.e. suitable for use in a cosmetic composition. Suitable polyamides include Polyamide-3 and Polyamide-8 (these being the INCI names). Polyamide-8 is a copolymer of ethylenediamine, neopentyl glycol and hydrogenated dilinoleic acid end-blocked with stearyl alcohol. Polyamide-3 is a polymer formed by the condensation of Dilinoleic Acid (q.v.), ethylenediamine, polypropylene glycol diamine end-capped with PEG/PPG-32/10 aminopropyl methyl ether. The polyamide component (a) is an oil-structuring polymer, so aids the formulation of the oil.

The cosmetic agent may include only one polyamide or a mixture of two or more different polyamides. The cosmetic agent may include a mixture of Polyamide-3 or Polyamide-8 with one or more other polyamides. Preferably, the cosmetic agent includes Polyamide-8 with one or more other polyamides. More preferably, the only polyamide present is Polyamide-8.

A preferred commercial product to provide the Polyamide-8 is OleoCraft LP-20 supplied by Croda.

Suitably, the total amount of the or each polyamide is from about 0.5 to about 30 wt %, relative to the total weight of the cosmetic agent. More suitably, the total amount of the or each polyamide is from about 1 to about 15 wt %, preferably from about 2 to about 10 wt %, more preferably from about 2 to about 5 wt %, relative to the total weight of the cosmetic agent.

As component (b), the cosmetic agents disclosed herein include at least one ester.

The or each ester is suitably an alkoxylated alcohol.

Suitable esters are C₆-C₃₀ fatty acids with C₂-C₃₀ alcohols, diols or polyols.

Examples of fatty acid components used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachinic acid, gadoleic acid, behenic acid and erucic acid and mixtures thereof. Examples of the alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof

The monoesters of fatty acids with alcohols with 2 to about 24 C atoms are preferred. A particularly preferred such monoester is isopropyl isostearate.

Another preferred ester (to be used alone or preferably in combination with one or more different esters) is neopentyl glycol diheptanoate.

The C₆-C₃₀ fatty acid may be unsaturated. A preferred ester of an unsaturated C₆-C₃₀ fatty acids with a C₂-C₃₀ alcohol is Heptyl Undecylenate.

Alternative esters for inclusion in the cosmetic agents disclosed herein include isopropyl myristate (Rilanit® IPM), isononanoic acid C16-18 alkyl esters (Cetiol® SN), 2-ethylhexyl palmitate (Cegesoft® 24), stearic acid 2-ethylhexyl esters (Cetiol® 868), cetyloleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl esters (Cetiol® A), di-n-butyl adipate (Cetiol® B), myristyl myristate (Cetiol® MM), cetearyl isononanoate (Cetiol® SN), oleic acid decyl esters (Cetiol® V).

A further preferred ester is PPG-3 Benzyl Ether Myristate.

A further preferred ester is a transester such as Shea Butter Ethyl Esters.

Suitably, the ester component in the cosmetic agent is isopropyl isostearate, optionally in combination with one or more different esters. Preferably, isopropyl isostearate is present in combination with 2, 3 or 4 different esters.

Suitably, the ester component in the cosmetic agent is neopentyl glycol diheptanoate, optionally in combination with one or more different esters. Preferably, neopentyl glycol diheptanoate is present in combination with 2, 3 or 4 different esters.

Suitably, the ester component in the cosmetic agent is heptyl undecylenate, optionally in combination with one or more different esters. Preferably, heptyl undecylenate is present in combination with 2, 3 or 4 different esters.

Suitably, the ester component in the cosmetic agent is PPG-3 Benzyl Ether Myristate, optionally in combination with one or more different esters. Preferably, PPG-3 Benzyl Ether Myristate is present in combination with 2, 3 or 4 different esters.

Suitably, the ester component in the cosmetic agent is Shea Butter Ethyl Esters, optionally in combination with one or more different esters. Preferably, Shea Butter Ethyl Esters is present in combination with 2, 3 or 4 different esters.

Thus, the cosmetic agents preferably comprise at least two esters selected from:

### Isopropyl Isostearate

### Neopentyl Glycol Diheptanoate

### Heptyl Undecylenate

### PPG-3 Benzyl Ether Myristate

### Shea Butter Ethyl Esters

The cosmetic agents more preferably comprise at least three esters selected from:

### Isopropyl Isostearate

### Neopentyl Glycol Diheptanoate

### Heptyl Undecylenate

### PPG-3 Benzyl Ether Myristate

### Shea Butter Ethyl Esters

Suitable combinations of esters include PPG-3 Benzyl Ether Myristate and Neopentyl Glycol Diheptanoate, together with one or more of Shea Butter Ethyl Esters, Isopropyl Isostearate and Heptyl Undecylenate.

A particularly preferred combination of esters is:

### PPG-3 Benzyl Ether Myristate

### Neopentyl Glycol Diheptanoate

### Shea Butter Ethyl Esters

### Isopropyl Isostearate

Another particularly preferred combination of esters is:

### PPG-3 Benzyl Ether Myristate

### Neopentyl Glycol Diheptanoate

### Heptyl Undecylenate.

Alternative esters may be selected from the group of dicarboxylic acid esters, carbonate esters and diol esters. These are understood to be:

dicarboxylic acid esters such as di-n-butyl adipate, di-(2-ethylhexyl) adipate, di-(2-ethylhexyl) succinate and di-isotridecylacelaate and
diol esters such as ethylene glycol dioleate, ethylene glycol-di-isotridecanoate, propylene glycol-di(2-ethylhexanoate), propylene glycol-di-isostearate, propylene glycol-di-pelargonate, butanediol-di-isostearate, neopentyl glycol dicaprylate, and
symmetrical, asymmetrical or cyclic esters of carbonic acid with fatty alcohols, such as glycerol carbonate or dicaprylyl carbonate (Cetiol® CC).

Suitable esters include carbonic acid with C6-12 alkanols, in particular C7-9 alkanols. The esters may particularly be symmetrical carbonic acid esters with alkanols, especially dicaprylyl carbonate (carbonic acid esters with n-octyl alcohol).

Further, suitable esters include tri-fatty acid esters of saturated and/or unsaturated linear and/or branched fatty acids with glycerol. Such esters may be selected from tri-fatty acid esters of saturated linear and/or branched C6-12 carboxylic acids with glycerol. Linear C6-12 carboxylic acids, in particular C8-10 carboxylic acids, may be used. A suitable mixture is of tri-fatty acid esters of caprylic acid and capric acid with glycerol, for example in a ratio by weight of caprylic acid triglyceride to capric acid triglyceride of from about 3:1 to about 1:1.

Suitably, the total amount of the or each ester is from about 5 to about 60 wt %, relative to the total weight of the cosmetic agent. Where the cosmetic agentcomprises only one ester, it will be understood that the total amount refers to the amount of the ester, relative to the total weight of the cosmetic agent. Where the cosmetic agentcomprises more than one ester, it will be understood that the total amount refers to the combined amounts of the esters present, relative to the total weight of the cosmetic agent. Preferably, the total amount of the or each ester is from about 5 to about 45 wt %, more preferably from about 10 to about 35 wt %, relative to the total weight of the cosmetic agent.

As component (c), the cosmetic agents disclosed herein include either Ricinus Communis (Castor) Seed Oil or isostearyl alcohol or a mixture thereof.

The cosmetic agents disclosed herein may further include at least one additional natural oil. It will be understood that the “additional” natural oil, if present, is a natural oil other than Ricinus Communis (Castor) Seed Oil.

The or each additional natural oil may be selected from, Crambe Abyssinica Seed Oil, amaranth seed oil, apricot kernel oil, argan oil, avocado oil, babassu oil, cottonseed oil, borage seed oil, camelina oil, safflower oil, peanut oil, pomegranate seed oil, grapefruit seed oil, hemp oil, hazelnut oil, elderberry seed oil, blackcurrant seed oil, jojoba oil, cocoa butter, linseed oil, Macadamia nut oil, corn oil, almond oil, manila oil, evening primrose oil, olive oil, orange oil, palm oil, peach kernel oil, rapeseed oil, rice bran oil, sea buckthorn fruit pulp oil, sea buckthorn seed oil, sesame oil, shea butter, soybean oil, sunflower oil, grape seed oil, walnut oil, wheat germ oil, wild rose oil, and the liquid parts of coconut oil.

The cosmetic agents disclosed herein may further include at least one additional fatty alcohol. It will be understood that the “additional” fatty alcohol, if present, is a fatty alcohol other than isostearyl alcohol. The additional fatty alcohol may be selected from isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and mixtures thereof. Preferably, component (c) includes or consists of Isostearyl Alcohol. Component (c) may be a mixture of Isostearyl Alcohol and Ricinus Communis (Castor) Seed Oil.

Suitably, the total amount of component (c) is from about 0.1 to about 10 wt %, relative to the total weight of the cosmetic agent. Preferably, the total amount of component (c) is from about 1 to about 8 wt %, preferably from about 2 to about 7 wt %, relative to the total weight of the cosmetic agent.

As component (d), the cosmetic agents disclosed herein include at least one hydrocarbon.

The hydrocarbons are preferably alkanes, suitably one or more alkanes selected from the group of isodecane, isoundecane, isododecane, isotridecane, and isotetradecane.

The cosmetic agent may include only one alkane or a mixture of two or more different alkanes. The cosmetic agent may include a mixture of isododecane with one or more other alkanes. Preferably, the hair oil includes isododecane present as the only alkane (falling under group (b)).

Suitably, the total amount of the or each hydrocarbon is from about 30 to about 80 wt %, relative to the total weight of the cosmetic agent. More suitably, the total amount of the or each hydrocarbon is from about 40 to about 70 wt %, relative to the total weight of the cosmetic agent.

An alkane that is particularly preferred as contemplated herein is commercially available under the name PUROLAN IDD (Isododecan). The alkane may be present in a commercial product as a mixture with an ester. For example, a preferred commercial product according to the present disclosure is LexFeel D5, which is a mixture of Neopentyl Glycol Diheptanoate and Isododecane.

The cosmetic agent as contemplated herein can be adapted to the particular hair quality by the selection of suitable amounts of the components disclosed herein. Formulations for the treatment of fine, normal or thick hair can thus be obtained selectively.

By use of the cosmetic agent as contemplated herein it is possible to dispense with organosilicon compounds, in particular silicones, in the cosmetic agent. In the context of the present disclosure, “silicone-free” is taken to mean that the cosmetic agent is free from organosilicon compounds, in particular free from silicones, trisiloxanes, and silicone oils.

The cosmetic agent is preferably present in the form of a hair oil. Particularly preferably, the hair oil is applied by a dispenser onto the user's hand. Thus, the packaging is adapted based on that method of application, as would be well understood by the skilled person. The user then distributes the hair oil from their hand/hands to the hair length and tips. The user may spread the hair oil on their hand/hands before distribution. The user may pump the hair oil into their hand, so the dispenser may include a pump. The dispenser may also include a pipette to enable the oil to be dispensed from the dispenser to the hand. The dispenser may also include a droplet inlay.

The cosmetic agent may alternatively be applied in the form of a sprayed hair oil and is sprayed onto the hair. The spray can be a pump spray, or the spraying can be achieved with the aid of propellants. Suitable propellants (propellant gases) are propane, propene, n-butane, iso-butane, iso-butene, n-pentane, pentene, iso-pentane, iso-pentene, methane, ethane, dimethylether, nitrogen, air, oxygen, nitrous oxide, 1,1,1,3-tetrafluoroethane, heptafluoro-n-propane, perfluoroethane, monochlorodifluoromethane, 1,1-difluoroethane, more specifically either individually or in combination. Hydrophilic propellant gases such as carbon dioxide, can also be used advantageously, if the proportion of hydrophilic gases is selected to be low and a lipophilic propellant gas (for example propane/butane) is present in excess. Propane, n-butane, isobutane, and mixtures of said propellant gases are particularly preferred. It has emerged that the use of n-butane as the sole propellant gas can be particularly preferred. Vessels made of metal (aluminium, tinplate, tin), protected or non-splintering plastic, or glass that is externally coated with plastic may be used as compressed gas containers; compressive strength and breaking strength, corrosion resistance, ease of filling, as well as aesthetic aspects, handling, printability, etc., play a role in their selection.

The present disclosure thus also relates to the use of the cosmetic agent for the care of keratin fibres.

A further subject of the present application is therefore a method for the treatment of keratin fibres, exemplified in that a cosmetic agent as contemplated herein is applied to the dried and/or damp keratin fibres.

The cosmetic agent as contemplated herein is preferably used directly before, during or after an oxidative or surfactant hair treatment. In the context of the present disclosure, the expression “directly before the oxidative or surfactant hair treatment” is understood to mean an application that directly follows the oxidative or surfactant hair treatment, wherein the hair treatment agent as contemplated herein has been rinsed beforehand from the hair or preferably has been left on the hair, and the hair is preferably still wet.

The expression “after the oxidative or surfactant hair treatment” in the context of the present disclosure is understood to mean an application which either directly follows the oxidative or surfactant hair treatment, wherein the hair treatment agent as contemplated herein is applied to the preferably still wet, towel-dried hair once the agent with oxidative or surfactant effect has been rinsed out, or is applied to the dry or wet hair only after several hours or days. In both cases the hair treatment agent as contemplated herein can be rinsed out again after a reaction time of a few seconds up to about 45 minutes, or can remain fully on the hair.

A further subject of the present application is the use of a cosmetic agent as contemplated herein for the care of keratin fibres. What is claimed here in particular is the use of a cosmetic agent as contemplated herein

to improve the wet and dry combability of keratin fibres,
to improve the shine of keratin fibres,
to improve the feel of keratin fibres,
to improve the regeneration of keratin fibres.

Combability is understood as contemplated herein to mean both the combability of the wet fibres and the combability of the dry fibres. The combing work involved, or the force applied during the process of combing a collective of fibres, is used as a measure for combability. The measurement parameters can be assessed in a sensory manner by a person skilled in the art or can be quantified using measuring devices.

Besides the active substances described above, the cosmetic agents as contemplated herein can contain further ingredients. The group of these further ingredients includes, in particular, the cosmetically effective active substances, auxiliaries and additives.

A first group of optional ingredients is formed by the additional substances that form oil bodies. Examples of these include di-n-alkyl ethers with a total of between about 12 and about 36 C atoms, for example:

di-n-alkyl ethers with a total between about 12 and about 36 C atoms, in particular about 12 to about 24 C atoms, such as di-noctyl ether, di-n-decyl ether, di-n-nonyl ether, di-n-undecyl ether, di-n-dodecyl ether, n-hexyl-noctyl ether, n-octyl-n-decyl ether, n-decyl-n-undecyl ether, n-undecyl-n-dodecyl ether and n- hexyl-n-undecyl ether and di-tert-butyl ether, di-iso-pentyl ether, di-3-ethyldecyl ether, tert.- butyl-n-octyl ether, iso-pentyl-n-octyl ether and 2-methyl-pentyl-n-octyl ether. The compounds 1,3-di-(2-ethyl-hexyl)-cyclohexane (Cetiol® S) and di-n-octyl ether (Cetiol® OE) obtainable as commercial products can be preferred

The cosmetic agent as contemplated herein can also contain fatty acid partial glycerides, that is to say monoglycerides, diglycerides and technical mixtures thereof, instead of component e) or as additional constituent. With use of technical products, small amounts of triglycerides may still be contained due to the production process. The partial glycerides preferably confirm to the following formula,

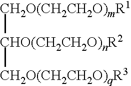

in which R¹, R² and R³ independently of one another stand for hydrogen or for a linear or branched, saturated and/or unsaturated acyl group with about 6 to about 22, preferably about 12 to about 18 carbon atoms, with the provision that at least one of these groups stands for an acyl group and at least of these groups stands for hydrogen. The sum (m+n+q) stands for 0 or numbers from about 1 to about 100, preferably for 0 or from about 5 to about 25. R¹ preferably stands for an acyl group, and R² and R³ preferably stand for hydrogen, and the sum (m+n+q) is 0. Typical examples are mono- and/or diglycerides based on caproic, caprylic acid, 2-ethylhexanoic acid, capric acid, lauric acid, isotridecanoic acid, myristic acid, palmitic acid, palmoleic acid, stearic acid, isostearic acid, oleic acid, elaidic acid, petroselinic acid, linoleic acid, linolenic acid, elaeostearic acid, arachinic acid, gadoleic acid, behenic acid and erucic acid and mixtures thereof. Oleic acid monoglycerides are preferably used.

Besides the active substances described above, the cosmetic agent as contemplated herein can contain further ingredients. The group of these further ingredients includes, in particular, the cosmetically effective active substances, auxiliaries and additives.

Suitable active substances, auxiliaries and additives are in particular additional care substances. The agent for example can contain at least one protein hydrolysate and/or one of the derivatives thereof as care substance. Protein hydrolysates are product mixtures that are obtained by acid-catalysed, base-catalysed or enzymatically catalysed breakdown of proteins. The term “protein hydrolysates” is understood as contemplated herein to also mean total hydrolysates and also individual amino acids and derivatives thereof as well as mixtures of different amino acids. The molecular weight of the protein hydrolysates usable as contemplated herein lies between about 75, the molecular weight for glycine, and about 200,000 daltons, and the molecular weight is preferably from about 75 to about 50,000 daltons, and very particularly preferably from about 75 to about 20,000 daltons.

As care substance, the agent as contemplated herein can also contain at least one vitamin, a provitamin, a vitamin precursor and/or one of the derivatives thereof. Here, vitamins, provitamins and vitamin precursors that are usually assigned to the groups A, B, C, E, F and H are preferred as contemplated herein.

Similarly to the addition of glycerol and/or propylene glycol, the addition of panthenol increases the flexibility of the polymer film formed with application of the agent as contemplated herein.

As care substance, the agents as contemplated herein can also contain at least one plant extract, but also monosaccharides or oligosaccharides and/or lipids.

The cosmetic agent disclosed herein may also include a perfume. Suitably, a perfume is included in an amount ranging from about 0.1 to about 0.8% by weight, preferably from about 0.2 to about 0.6%, relative to the weight of the cosmetic agent.

Particularly preferred cosmetic agents as contemplated herein are preferably formulated as application mixtures with a low water content. The water content of preferred cosmetic agents, in relation to the total weight of said cosmetic agents, is less than about 10% by weight, preferably less than about 5.0% by weight, particularly preferably less than about 1.0% by weight, and in particular less than about 0.1% by weight, wherein very particularly preferred cosmetic agents contain no water.

Suitably, the cosmetic agent disclosed herein has any viscosity that renders it suitable for application to the hair. Viscosity of the cosmetic agent disclosed herein may be measured in centipoise (cps). The cosmetic agent disclosed herein may have a viscosity in the range of from about 0.8 cps to about 500,000 cps. In one example, a hair oil as disclosed herein is offered as a dispensable liquid with a viscosity in the range of from about 300 to about 5,000 cps, such as from about 1,000 cps to about 4,000 cps.

### Methods

The present specification is also directed to a method for using a cosmetic agent as contemplated herein. When the cosmetic agent is a hair oil, the method includes applying the hair oil as disclosed herein to the hair. The method includes dispensing the hair oil as disclosed herein onto the user's hand, and from the user's hand onto the hair. The method may additionally include rubbing the hair. The method may include the user spreading the hair oil on their hand or hands before application to the hair.

The method for using a cosmetic agent as disclosed herein may also include additional, optional steps. In one example, such steps may include air drying the hair, drying the hair using heat, styling the hair, and any other suitable step that is known to an individual skilled in the art of hair treatment.

The present disclosure may be exemplified by the following statements.

A silicone-free cosmetic agent comprising:

a) one or more polyamides,
b) one or more esters,
c) Ricinus Communis (Castor) Seed Oil, isostearyl alcohol, or a mixture thereof, and
d) one or more hydrocarbons.

A cosmetic agent as contemplated herein, wherein the polyamide is Polyamide-3 or Polyamide-8.

A cosmetic agent as contemplated herein, wherein the polyamide is Polyamide-8.

A cosmetic agent as contemplated herein, wherein the polyamide is Polyamide-3.

A cosmetic agent as contemplated herein, wherein the total amount of the or each polyamide is from about 0.5 to about 30 wt %, relative to the total weight of the cosmetic agent.

A cosmetic agent as contemplated herein, wherein the total amount of the or each polyamide is from about 1 to about 15 wt %,

A cosmetic agent as contemplated herein, wherein the total amount of the or each polyamide is from about 2 to about 10 wt %.

A cosmetic agent as contemplated herein, wherein the total amount of the or each polyamide is from about 2 to about 5 wt %, relative to the total weight of the cosmetic agent.

A cosmetic agent as contemplated herein, wherein the or each ester is selected from esters of C₆-C₃₀ fatty acids with C₂-C₃₀ alcohols, diols or polyols.

A cosmetic agent as contemplated herein, wherein the ester includes or consists of isopropyl isostearate.

A cosmetic agent as contemplated herein, wherein the ester includes or consists of neopentyl glycol diheptanoate.

A cosmetic agent as contemplated herein, wherein the ester includes or consists of PPG-3 Benzyl Ether Myristate.

A cosmetic agent as contemplated herein, wherein the ester includes or consists of Shea Butter Ethyl Esters.

A cosmetic agent as contemplated herein, wherein the ester includes or consists of Heptyl Undecylenate.

A cosmetic agent as contemplated herein, wherein the ester includes or consists of PPG-3 Benzyl Ether Myristate and Neopentyl Glycol Diheptanoate.

A cosmetic agent as contemplated herein, wherein the ester is a mixture of PPG-3 Benzyl Ether Myristate, Shea Butter Ethyl Esters, Isopropyl Isostearate and Neopentyl Glycol Diheptanoate.

A cosmetic agent as contemplated herein, wherein the ester is a mixture of PPG-3 Benzyl Ether Myristate, Heptyl Undecylenate and Neopentyl Glycol Diheptanoate.

A cosmetic agent as contemplated herein, wherein the total amount of the or each ester is from about 5 to about 60 wt %, relative to the total weight of the cosmetic agent.

A cosmetic agent as contemplated herein, wherein the total amount of the or each ester is from about 5 to about 45 wt %.

A cosmetic agent as contemplated herein, wherein the total amount of the or each ester is from about 10 to about 35 wt %, relative to the total weight of the cosmetic agent.

A cosmetic agent as contemplated herein, wherein component (c) is Ricinus Communis (Castor) Seed Oil.

A cosmetic agent as contemplated herein, wherein component (c) is Isostearyl Alcohol.

A cosmetic agent as contemplated herein, wherein component (c) is a mixture of Ricinus Communis (Castor) Seed Oil and Isostearyl Alcohol.

A cosmetic agent as contemplated herein, wherein the total amount of component (c) is from about 0.1 to about 10 wt %, relative to the total weight of the cosmetic agent.

A cosmetic agent as contemplated herein, wherein the total amount of component (c) is from about 1 to about 8 wt %.

A cosmetic agent as contemplated herein, wherein the total amount of component (c) is from about 2 to about 7 wt %, relative to the total weight of the cosmetic agent.

A cosmetic agent as contemplated herein, wherein the or each hydrocarbon is an alkane.

A cosmetic agent as contemplated herein, wherein the hydrocarbon is selected from isodecane, isoundecane, isododecane, isotridecane, isotetradecane, C12-17 Alkane and mixtures thereof.

A cosmetic agent as contemplated herein, wherein the hydrocarbon includes or consists of isododecane.

A cosmetic agent as contemplated herein, wherein the hydrocarbon includes or consists of C12-17 Alkane.

A cosmetic agent as contemplated herein, wherein the hydrocarbon is a mixture of isododecane and C12-17 Alkane.

A cosmetic agent as contemplated herein, wherein the total amount of the or each hydrocarbon is from about 30 to about 80 wt %, relative to the total weight of the cosmetic agent.

A cosmetic agent as contemplated herein, wherein the total amount of the or each hydrocarbon is from about 40 to about 70 wt %, relative to the total weight of the cosmetic agent.

A cosmetic agent as contemplated herein, in the form of a hair oil.

A cosmetic product comprising the cosmetic agent as contemplated herein.

A cosmetic product as contemplated herein, wherein the product comprises a pump dispenser.

A cosmetic product as contemplated herein, wherein the product comprises a pipette.

A cosmetic product as contemplated herein, wherein the product comprises a droplet inlay.

Use of a cosmetic agent or a cosmetic product as contemplated herein for the care of hair fibres.

A method for using a cosmetic agent as contemplated herein, or a cosmetic product as contemplated herein, the method comprising applying the cosmetic agent to the hair.

A method as contemplated herein, wherein the application comprises dispensing the cosmetic agent onto the user's hand, followed by application of the cosmetic agent from the user's hand to the hair.

### EXAMPLES

All specified amounts relate to parts by weight. The following formulations were provided with the use of known production methods.

### Example 1—in the Form of a Hair Oil

| Ingredient (INCI Name) | wt % |
|---|---|
| Polyamide-8 | 4.0 |
| PPG-3 Benzyl Ether Myristate | 20.0 |
| Shea Butter Ethyl Esters | 1.0 |
| Isopropyl Isostearate | 10.0 |
| Ricinus Communis (Castor) Seed Oil | 5.0 |
| Isododecane | 52.2 |
| Neopentyl Glycol Diheptanoate | 7.8 |
| | 100 |

### Example 2—in the Form of a Hair Oil

| Ingredient (INCI Name) | wt % |
|---|---|
| Polyamide-8 | 4.0 |
| PPG-3 Benzyl Ether Myristate | 10.0 |
| Heptyl Undecylenate | 5.0 |
| C12-17 Alkane | 45.0 |
| Isostearyl Alcohol | 5.0 |
| Isododecane | 21.3 |
| Neopentyl Glycol Diheptanoate | 9.4 |
| Perfume | 0.3 |
| | 100 |

For example:

SP Oleocraft LP-20 MBAL-PA-(MV) can be used as Polyamide-8
Crodamol STS-LQ-(MH) can be used as PPG-3 Benzyl Ether Myristate
Lipex Shea Light can be used as Shea Butter Ethyl Esters
CRODAMOL IPIS-LQ-(MV) can be used as Isopropyl Isostearate
Castor oil DAB/Ph.Eur. can be used as Ricinus Communis (Castor) Seed Oil
PUROLAN IDD can be used as Isododecane
LexFeel D5, which is a mixture of Neopentyl Glycol Diheptanoate and Isododecane, can be used

The exemplified hair oils were found to be advantageous in terms of silky hair feel, enhancement of shine/gloss, and beneficial smoothness, softness, and detangling.

It will be appreciated that the present disclosure may be modified within the scope of the appended claims.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A silicone-free cosmetic agent comprising:
a) one or more polyamides,
b) one or more esters,
c) Ricinus Communis (Castor) Seed Oil, isostearyl alcohol, or a mixture thereof, and
d) one or more hydrocarbons.

2. A cosmetic agent according to claim 1, wherein the polyamide is Polyamide-3 or Polyamide-8.

3. A cosmetic agent according to claim 1, wherein the total amount of each of the one or more polyamides is from about 0.5 to about 30 wt %, relative to the total weight of the cosmetic agent.

4. A cosmetic agent according to claim 1, wherein the one or more esters is selected from esters of C₆-C₃₀ fatty acids with C₂-C₃₀ alcohols, diols or polyols.

5. A cosmetic agent according to claim 1, wherein the one or more esters comprises a mixture of two or more of isopropyl isostearate, neopentyl glycol diheptanoate, PPG-3 Benzyl Ether Myristate, Shea Butter Ethyl Esters and Heptyl Undecylenate.

6. A cosmetic agent according to claim 1, wherein the one or more esters is a mixture of PPG-3 Benzyl Ether Myristate, Shea Butter Ethyl Esters, Isopropyl Isostearate and Neopentyl Glycol Diheptanoate or a mixture of PPG-3 Benzyl Ether Myristate, Heptyl Undecylenate and Neopentyl Glycol Diheptanoate.

7. A cosmetic agent according to claim 1, wherein the total amount of each of the one or more esters is from about 5 to about 60 wt %.

8. A cosmetic agent according to claim 1, wherein the total amount of component (c) is from about 0.1 to about 10 wt %, relative to the total weight of the cosmetic agent.

9. A cosmetic agent according to claim 1, wherein each of the one or more hydrocarbons is an alkane.

10. A cosmetic agent according to claim 1, wherein each of the one or more hydrocarbons comprises isododecane, C12-17 Alkanes, or combinations thereof

11. A cosmetic agent according to claim 1, wherein the total amount of each of the one or more hydrocarbons is from about 30 to about 80 wt %.

12. A cosmetic agent according to claim 1, that is a hair oil.

13. A cosmetic agent according to claim 1, wherein the total amount of each of the one or more polyamides is from about 1 to about 15 wt %, relative to the total weight of the cosmetic agent.

14. A cosmetic agent according to claim 1, wherein the total amount of each of the one or more polyamides is from about 2 to about 10 wt %, relative to the total weight of the cosmetic agent.

15. A cosmetic agent according to claim 1, wherein the total amount of each of the one or more polyamides is from about 2 to about 5 wt %, relative to the total weight of the cosmetic agent.

16. A cosmetic agent according to claim 2, wherein the total amount of each of the one or more polyamides is from about 1 to about 15 wt %, relative to the total weight of the cosmetic agent.

17. A cosmetic agent according to claim 2, wherein the total amount of each of the one or more polyamides is from about 2 to about 10 wt %, relative to the total weight of the cosmetic agent.

18. A cosmetic agent according to claim 2, wherein the total amount of each of the one or more polyamides is from about 2 to about 5 wt %, relative to the total weight of the cosmetic agent.

19. A cosmetic agent according to claim 1,
wherein the one or more polyamides is Polyamide-3 or Polyamide-8,
wherein the total amount of the Polyamide-3 or Polyamide-8 is from about 2 to about 5 wt %, relative to the total weight of the cosmetic agent,
wherein the one or more esters is a mixture of PPG-3 Benzyl Ether Myristate, Shea Butter Ethyl Esters, Isopropyl Isostearate and Neopentyl Glycol Diheptanoate or a mixture of PPG-3 Benzyl Ether Myristate, Heptyl Undecylenate and Neopentyl Glycol Diheptanoate,
wherein the total amount of each of the one or more esters is from about 10 to about 35 wt %,
wherein the total amount of component (c) is from about 2 to about 7 wt %, relative to the total weight of the cosmetic agent,
wherein the hydrocarbon comprises isododecane, C12-17 Alkanes, or combinations thereof, and is present in an amount of from about 40 to about 70 wt %.
